(19) United States
(12) Patent Application Publication
(10) Pub. No.: US 2020/0239393 A1
(43) Pub. Date: Jul. 30, 2020

(54) METHOD FOR CHEMICAL CONVERSION OF SUGARS OR SUGAR ALCOHOLS TO GLYCOLS

(71) Applicants: thyssenkrupp Industrial Solutions AG, Essen (DE); thyssenkrupp AG, Essen (DE)

(72) Inventors: Christoph GLOTZBACH, Herford (DE); Steffen SCHIRRMEISTER, Mülheim an der Ruhr (DE); Regina PALKOVITS, Aachen (DE); Peter HAUSOUL, Landgraaf (NL); Anna Katharina BEINE, Aachen (DE)

(73) Assignees: thyssenkrupp Industrial Solutions AG, Essen (DE); thyssenkrupp AG, Essen (DE)

(21) Appl. No.: 16/492,808

(22) PCT Filed: Mar. 15, 2018
(86) PCT No.: PCT/DE2018/100236

§ 371 Date: Sep. 10, 2019

(30) Foreign Application Priority Data
Mar. 15, 2017 (DE) 10 2017 204 322.9

(51) Int. Cl. C07C 29/60; B01J 35/02; B01J 37/16

(52) U.S. Cl. CPC C07C 29/60; C07C 31/18; B01J 37/16; B01J 35/023

(57) ABSTRACT

Methods for chemically converting sugars or sugar alcohols into polyols/glycols, wherein the sugars or sugar alcohols are converted by means of hydrogenolysis in the presence of a catalyst comprising at least one metal and on a carbon support, wherein a nitrogen-doped carbon support is used as a catalyst support. The disclosure provides methods for chemically converting sugars or sugar alcohols into glycols which permits the preparation of glycols with higher selectivity and reduces the formation of lactic acid as a by-product.

The present invention relates to a method for chemically converting sugars or sugar alcohols into polyols/glycols.

The preparation of basic and fine chemicals and also the extraction of energy from petroleum, coal and natural gas is prior art. However, these carbon sources are often difficult to access and non-renewable. The extraction and processing of fossil fuels is energy intensive and produces considerable quantities of greenhouse gases. The use of biomass represents a sustainable and CO₂-neutral alternative. Vegetable biomass is decomposed by means of fermentation or other methods. Further workup of the cleavage products is necessary for creation of value from the constituents of the biomass. Accordingly, the present invention relates to the chemical transformation of bio-based sugars and sugar alcohols into glycols.

In the conventional method, ethylene glycol is prepared from ethylene, which is converted into ethylene oxide and then hydrated to give ethylene glycol. Propylene glycol is customarily prepared by hydrating propylene oxide.

The conversion of sugars and sugar alcohols into glycols is likewise known from the literature. By way of example, WO 03/035593 A1 describes a method for converting 5-C sugars and sugar alcohols using hydrogen at elevated temperatures of more than 120° C., basic pH and in the presence of a rhenium-containing catalyst that additionally contains nickel. In this case there is hydrogenolysis both of C—C bonds and of CO bonds, with the result that the 5-C sugars and sugar alcohols are cleaved and, via intermediate steps, propylene glycol (CH₃—CHOH—CH₂OH) is formed as product. Alternative metallic catalysts mentioned are also Ru, Pt, Pd, Ir and Rh.

Glycerol and lactic acid are formed as by-products in the hydrogenolysis to ethylene glycol and propylene glycol. The challenge in the aforementioned method is therefore that of suppressing lactic acid formation and optimizing the glycol selectivity.

Nitrogen-containing carbon supports and carbon nanotubes (CNTs) can be obtained via various synthesis routes and comprise different nitrogen contents depending on the production. They are mainly used for the catalysis of oxidation reactions, gas adsorption and in electrochemistry.

US 2010/0276644 A1 describes a method for preparing nitrogen-doped carbon nanotubes in which firstly a metal is precipitated out from a solution of a metal salt in a solvent, with the result that a suspension is obtained from which the solid is removed to give a heterogeneous metal catalyst. This catalyst is introduced into a fluidized bed in which it is allowed to react with a carbon- and nitrogen-containing material, as a result of which the nitrogen-doped carbon nanotubes are obtained. The metal salt used as a starting point is preferably a salt of cobalt, manganese, iron or molybdenum. The heterogeneous metal catalyst additionally comprises Al₂O₃ and MgO. The carbon- and nitrogen-containing material is, for example, an organic compound that is in a gaseous state and which can by way of example be selected from acetonitrile, dimethylformamide, acrylonitrile, propionitrile, butyronitrile, pyridine, pyrrole, pyrazole, pyrrolidine and piperidine. The stated US specification 2010/0276644 A1 proposes the use of the carbon nanotubes as additives for mechanically reinforcing materials and also for increasing the electrical conductivity or thermal conductivity thereof. By way of example, the nitrogen-doped carbon nanotubes are suitable for the production of conductor paths, batteries or illumination devices, or as a storage medium for hydrogen or lithium in membranes. In addition, mention is made of applications in fuel cells or in the medical sector as frameworks for controlling the growth of cellular tissue. In this document, therefore, a very broad spectrum of applications in a very wide variety of fields is mentioned.

WO 2016/119568 A1 describes a heteroatom-containing nanocarbon material and also the production thereof. The material contains up to 2% by weight of nitrogen and 1% to 6% by weight of oxygen. The nanocarbon material described therein is intended to have good catalytic properties in the dehydrogenation of hydrocarbons.

The problem addressed by the present invention is that of providing a method for chemically converting sugars or sugar alcohols into glycols, having the features of the kind stated in the introduction, which permits the preparation of glycols with higher selectivity and reduces the formation of lactic acid as by-product.

The solution to the aforementioned problem is provided by a method of the type mentioned in the introduction and having the features of claim 1.

According to the invention, provision is made for a nitrogen-doped carbon support to be used as catalyst support.

One preferred development of the inventive solution to the problem provides for a nitrogen-doped carbon support, especially nitrogen-doped activated carbon, to be used as catalyst support. As an alternative, for example, nitrogen-doped carbon black can be used as carbon support.

A nitrogen-doped carbon support is understood within the context of the present invention to be:

A carbon support, the surface of which includes nitrogen doping. This nitrogen-doped carbon support can be produced both by means of suitable precursor materials during the production of the carbon support itself and subsequently, for example, by means of reductive methods. Two possible methods that were used in the context of the present invention are described in the examples.

According to an alternative preferred variant of the method according to the invention, nitrogen-doped carbon nanotubes are used as catalyst support.

Nitrogen-doped carbon nanotubes are defined according to the present invention as follows:

Cylindrical carbon hollow bodies having a diameter of 3 to 90 nm which were doped with nitrogen prior to, during or after production of the carbon hollow body.

A base is preferably used as co-catalyst. Within the context of the present invention, the following bases in particular can be considered here:

All alkali metal hydroxides, especially sodium hydroxide (NaOH), potassium hydroxide (KOH) and lithium hydroxide (LiOH).

All alkaline earth metal hydroxides, especially magnesium hydroxide (Mg(OH)₂), calcium hydroxide (Ca(OH)₂), strontium hydroxide (Sr(OH)₂) and barium hydroxide (Ba(OH)₂).

If, in the method according to the invention, a sugar is the starting point, there is a two-stage process in which firstly the sugar is hydrogenated in a manner known per se to give the sugar alcohols and subsequently in a second step, using a catalyst, hydrogenolysis of the sugar alcohols, which are formed during the hydrogenation of the sugars, to give the polyols takes place.

It is also generally possible here to perform both reaction processes mentioned in only one step, or in a reaction sequence in a reactor, so that the sugars are converted directly into the polyols. The yields are poorer in this variant since various mutually interfering reaction mechanisms take place in parallel.

The method according to the invention is useful in particular for the hydrogenation with subsequent hydrogenolysis of the following sugars and the resultant sugar alcohols:

C5 sugars, for example the compounds mentioned hereafter:

ribose, arabinose, xylose, lyxose;

C5 sugar alcohols, for example the compounds mentioned hereafter:

ribitol, arabitol, xylitol, lyxitol;

C6 and other sugars and sugar alcohols, for example:

allose, altrose, glucose, mannose, gulose, idose, galactose, talose, allitol, talitol, sorbitol, mannitol, iditol, fucitol, galactitol, erythritol, threitol, glycerol.

When hydrogenolyzing C5 sugars, the products mentioned hereafter are formed:

xylitol, ribitol, arabitol, lyxitol.

When hydrogenolyzing C5 sugar alcohols, the cleavage products mentioned hereafter are formed: glycerol, ethylene glycol, propylene glycol, lactic acid, glycolic acid and also under certain reaction conditions erythritol, and anhydroxylitol.

According to one development of the invention, the conversion is preferably effected at a reaction temperature in the range from approximately 170° C. to approximately 200° C.

The aforementioned temperature range is advantageous since, if lower temperatures are chosen, reaction takes place very slowly or not at all.

If higher temperatures are chosen, deoxygenation and decarbonylation reactions, and also cyclizations, inter alia, are encountered to an increased degree. Considerably more by-products are formed, such as for example erythritol, threitol and anhydroxylitol.

According to one preferred development of the method according to the invention, the hydrogenolysis is effected at a hydrogen pressure in the range from approximately 50 bar to approximately 80 bar.

The aforementioned hydrogen pressures have been found to be particularly advantageous since, if lower pressures are chosen, more by-products are formed. Carbonyl formation is preferred.

If hydrogen pressures above the stated range are chosen, this has the disadvantage that firstly the reaction is difficult to implement industrially and secondly the conversion of the reactant is slowed considerably. By way of example, within the context of the present invention the catalyst can contain ruthenium and/or platinum and/or nickel as metal. The remaining elements of the platinum group (Os, Rh, Ir, Pd) and also Au, Ni, Cu, Fe and Co are additionally useful. In this case, the catalyst according to the invention can contain one or more of the stated metals.

The present invention is described in more detail below on the basis of exemplary embodiments with reference to the accompanying drawings. In the figures:

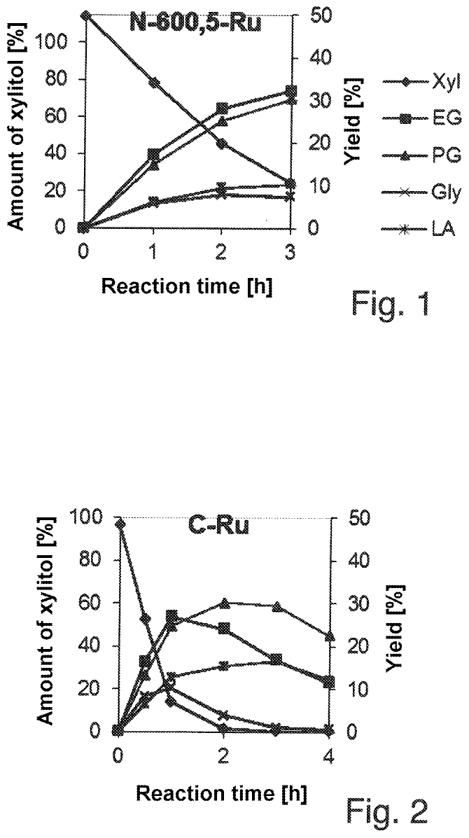

FIG. 1 shows the product formation over time for N-600,5-Ru (conditions: T=200° C., p(H₂)=80 bar, m(cat)=0.1563 g, m(xylitol)=1.50 g, m(Ca(OH)₂)=0.225 g, 15 ml H₂O);

FIG. 2 shows the product formation over time for C—Ru;

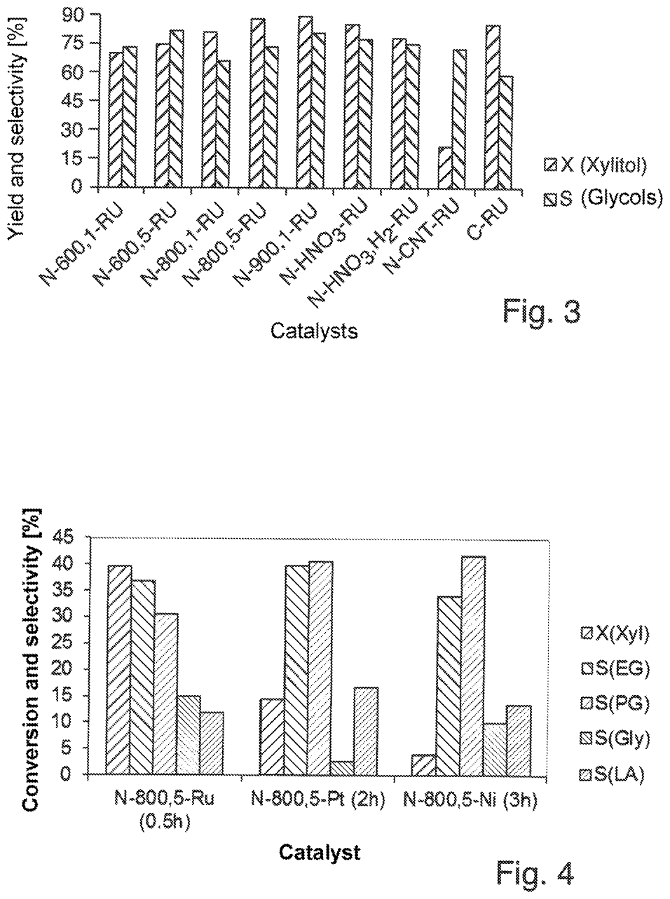

FIG. 3 shows a comparison of the catalysts (conditions: T=200° C., p(H₂)=80 bar, m(Ru)=0.01 g, m(xylitol)=1.50 g, m(Ca(OH)₂)=0.225 g, 15 ml H₂O);

FIG. 4 shows screening of metals on nitrogen-containing carbons (conditions: T=200° C., p(H₂) =80 bar, m(M)=5 mg, m(xylitol)=1.00 g, m(Ca(OH)₂)=0.150 g, 10 ml H₂O);

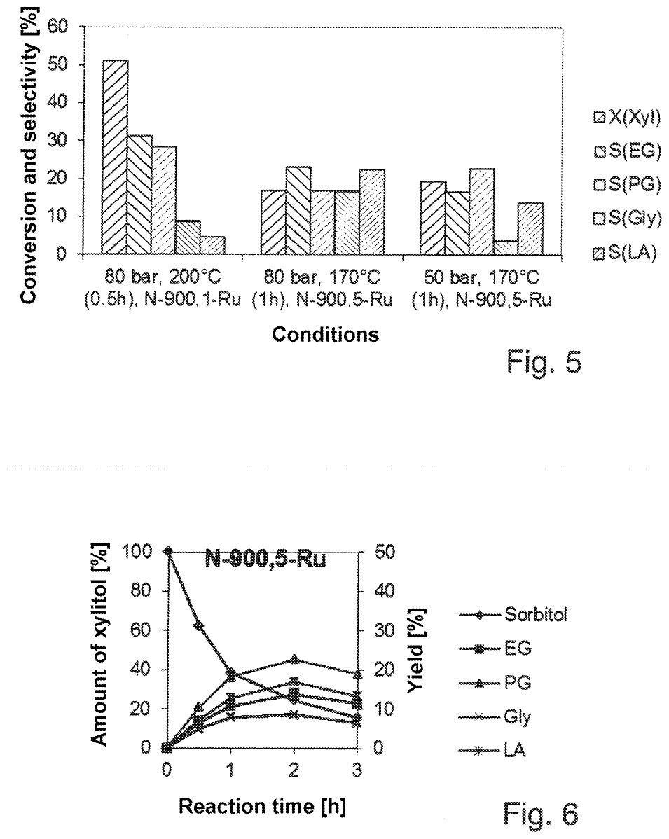

FIG. 5 shows the temperature and pressure variation for Ru/N-900-C (conditions: m(Ru)=5 mg, m(xylitol)=1 g, m(Ca(OH)₂)=0.150 g, 10 ml H₂O);

FIG. 6 shows the hydrogenolysis of sorbitol over N-900,5-Ru (conditions: T=200° C., p(H₂)=80 bar, m(Ru)=5 mg, m(sorbitol)=1.20 g, m(Ca(OH)₂)=0.150 g, 10 ml H₂O).

### EXAMPLE 1: PRODUCTION OF NITROGEN-CONTAINING CARBON SUPPORTS (N—C)

The example illustrates the production of nitrogen-doped carbon supports. 5 g of activated carbon are admixed with 35 ml of HNO₃ (30%) and refluxed for 8 h. The carbon is subsequently washed to neutral with water and dried. 1 g of the oxidized carbon is placed into a 50 ml autoclave charged with 8 bar of NH₃ and 52 bar of N₂. The autoclave is heated to 200° C. while stirring. The reduction of the carbon takes place over 4 h. For the carbon support described (N—HNO₃), a nitrogen content of 5.54% results according to CHN analysis. If the support is subsequently reduced further with hydrogen for 7 h at 350° C. (N—HNO₃, H₂), a nitrogen content of 4.65% results. In this way, however, the proportion of oxygen on the carbon is also reduced. The sum total of C, H and N is now 91.50%, whereas in the case of N—HNO₃ it is 84.62%.

A different way of doping carbon supports with nitrogen is reduction with gaseous ammonia. Temperatures of between 600 and 900° C. and times of 1 to 5 h are chosen for the reduction and various carbons are obtained. The use of commercial nitrogen-doped carbon nanotubes (N-CNT) is also possible. Prior to use, the NCNTs are heated under reflux with 10% by weight of HCl for 2 h. They are subsequently washed to neutral with water and dried. A summary of the nitrogen contents obtained for the various carbon supports is given in table 1.

### EXAMPLE 2: IMPREGNATION OF THE SUPPORT

The example illustrates the loading of a nitrogen-containing carbon support with a noble metal. Ruthenium is used by way of example. 500 mg of the carbon supports produced are each added, together with 75.72 mg of dichloro(p-cymene)ruthenium(II) dimer, to 145 ml of ethanol and coordinated in an oil bath under protective gas at 60° C. The coordination is terminated after 71 hours and the catalyst is filtered off. The maximum possible loading with ruthenium by this method is 5% by weight. The uncoordinated ruthenium in the solvent is analyzed by means of ICP MS and the loading of the catalyst is determined therefrom by calculation. The loading does not correlate with the nitrogen content and can be seen in table 1.

### EXAMPLE 3: HYDROGENOLYSIS OF XYLITOL

By way of example, the example illustrates the hydrogenolysis of sugars and sugar alcohols on the basis of the use of xylitol (Xyl). The hydrogenolysis is effected at 200° C. and 80 bar hydrogen pressure in a 50 ml autoclave. 1.50 g of xylitol, 0.225 g of Ca(OH)₂ and 15 ml of water are added to the autoclave. In addition, an amount of catalyst sufficient for there to be 7.5 mg of Ru in the reaction solution is added. For the catalyst N-800,1-Ru there is thus an amount of 0.1563 g, for Ru/C (C—Ru) there is 0.1500 g. The reaction was conducted over 3 to 4 h. Samples were taken at regular intervals in order to obtain kinetics. The product formation over time for N-600,5-Ru and C—Ru is shown in FIGS. 1 and 2. The desired products ethylene glycol (EG) and propylene glycol (PG) are formed as main products. Glycerol (Gly) and lactic acid (LA) are formed only in small amounts as by-products.

For all nitrogen-containing catalysts, EG (ethylene glycol) and PG (propylene glycol) are formed as main products under these conditions. The sum total of the two selectivities (S(Glycols)) in either case reaches above 67% for nitrogen-doped supports. However, for C—Ru, decomposition of the products can be observed in the case of a longer reaction time as a result of undesirable side-reactions (FIG. 2). A comparison of the catalysts is shown in FIG. 3. The maximum obtained glycol selectivity is 83% (N-600,5-Ru). Numerical values can be found in table 1.

TABLE 1
Prepared carbon supports, nitrogen content, metal loading and hydrogenolysis results.

| Carbon | Nitrogen | Ru loading/% | Reaction | S(EG)/ | S(PG)/ | X(Xyl)/ |
|---|---|---|---|---|---|---|
| support | content/% | by weight | time/h | % | % | % |
| N-600,1 | 0.49 | 4.43 | 3 | 38 | 36 | 70 |
| N-600,5 | 0.56 | 4.80 | 3 | 43 | 40 | 76 |
| N-800,1 | 1.64 | 3.36 | 1 | 37 | 30 | 52 |
| N-800,5 | 1.91 | 3.35 | 2 | 38 | 36 | 89 |
| N-900,1 | 1.41 | 1.36 | 3 | 41 | 41 | 94 |
| N-900,5 | 1.11 | 5.00 | — | — | — | — |
| N-HNO₃ | 5.54 | 4.80 | 2 | 38 | 40 | 86 |
| N-HNO₃,H₂ | 4.65 | 3.85 | 3 | 36 | 38 | 91 |
| N-CNT | 4.20 | 4.99 | 1 | 32 | 42 | 21 |
| C | 0.00 | 5.00 | 1 | 31 | 29 | 86 |

### EXAMPLE 4: SCREENING OF METALS ON NITROGEN-CONTAINING CARBONS

The metals Ni, Pt and Ru are compared. They were loaded onto the support N-800,5. Impregnation was effected in a manner equivalent to example 2. After loading, N-800,5-Pt and N-800,5-Ni were reduced in a stream of hydrogen. The reduction was effected at 350° C. for 7 h. The hydrogenolysis was effected in a manner equivalent to example 3. The results are presented in FIG. 4 and table 2. It is clearly apparent that the catalytic activity for N-800,5-Pt and N-800,5-Ni decreases, yet the selectivities for glycols obtained are unchanged and high.

TABLE 2
Screening of metals on nitrogen-containing carbons—
metal loading, hydrogenolysis results.

| | Metal | Reaction | | | |
|---|---|---|---|---|---|
| | loading/ | time/ | S(EG)/ | S(PG)/ | X(Xyl)/ |
| Catalyst | % | h | % | % | % |
| N-800, 5-Ru | 3.35 | 0.5 | 37 | 31 | 39 |
| N-800, 5-Pt | 4.99 | 2 | 40 | 41 | 14 |
| N-800, 5-Ni | 2.41 | 3 | 34 | 42 | 4 |

### EXAMPLE 5: COMPARISON WITH THE LITERATURE

The results of the hydrogenolysis of sugars and sugar alcohols from the present invention are compared hereafter with other catalysts and processes from the relevant prior art in respect of product selectivity and catalyst activity. The comparison is made under similar conditions and using similar substrates (see table 3).

TABLE 3
Comparison of the present invention with the prior art.

| | | | | Cat.: | T/ | p(H₂)/bar, | | | S(Glycols)/ |
|---|---|---|---|---|---|---|---|---|---|
| Ref. | Substrate | Catalyst | Base | Sub | ° C. | RT | t/h | X/% | % |
| [1] | sorbitol | Ni-Re/C | KOH | 1:10 | 220 | 83 | 4 | 56 | 46 |
| [2] | xylitol | Ru/C | KOH | 1:0.8 | 230 | 12 | — | 45 | 73 |
| [3] | xylitol | Ni-Re/C | KOH | 1:10 | 200 | 83 | — | 50 | 65 |
| * | xylitol | Ru/N-C | Ca(OH)₂ | 1:10 | 200 | 80 | 3 | 76 | 83 |

*present invention

[1] T. Werpy, J. Frye, A. Zacher, D. Miller, US20030119952 A1, 2003. [2] S. P. Chopade, D. J. Miller, J. E. Jackson, T. A. Werpy, J. G. Frye, Jr., A. H. Zacher, WO2001066499, 2001. [3] J. G. Frye, D. J. Miller, T. A. Werpy, A. H. Zacher, WO2003035593 B1, 2003.

### EXAMPLE 6: TEMPERATURE AND PRESSURE VARIATION FOR Ru/N—C

A variation of the temperature and pressure was conducted for the catalyst N-900-Ru. In addition to 200° C., 170° C. was also used, and in addition to 80 bar H₂, 50 bar H₂ was also used. The hydrogenolysis was conducted in a manner equivalent to example 3 and the results are presented in a comparative manner in FIG. 5 and table 4. The reaction proceeds more slowly for lower temperatures. It is difficult to compare the selectivities, since comparison at an identical conversion is not possible. Nevertheless, glycols remain the main products of the reaction.

TABLE 4
Prepared carbon supports, nitrogen content, metal loading and hydrogenolysis results.

| Catalyst | Reaction time/h | p/h | T/° C. | S(EG)/% | S(PG)/% | X(Xyl)/% |
|---|---|---|---|---|---|---|
| N-900,1-Ru | 0.5 | 80 | 200 | 31 | 28 | 51 |
| N-900,5-Ru | 1 | 80 | 170 | 23 | 17 | 17 |
| N-900,5-Ru | 1 | 50 | 200 | 17 | 23 | 20 |

### EXAMPLE 7: HYDROGENOLYSIS OF SORBITOL

The example illustrates the hydrogenolysis of sugars and sugar alcohols on the basis of the use of sorbitol (Sor). The hydrogenolysis is effected at 200° C. and 80 bar hydrogen pressure in a 50 ml autoclave. 1.197 g of sorbitol, 0.150 g of Ca(OH)₂ and 10 ml of water are added to the autoclave. In addition, an amount of catalyst sufficient for there to be 5 mg of Ru in the reaction solution is added. For the catalyst N-900,5-Ru there is thus an amount of 0.100 g. The reaction was conducted over 3 h. Samples were taken at regular intervals in order to obtain kinetics. The product formation over time for N-900,5-Ru is shown in FIG. 6. The selectivity for EG after 2 h of reaction is 18%, the selectivity for PG is 30%.

Comparative experiments were conducted within the context of the present invention in which carbon nanotubes that were not doped with nitrogen were used as catalyst supports. Here, conditions comparable to those in the aforementioned examples according to the invention were used. It was determined that the catalyst supports according to the invention are superior here, since they have an influence on the selectivities for the target products.

Comparative experiments were conducted within the context of the present invention in which activated carbon that was not doped with nitrogen was used as catalyst support. Here, conditions comparable to those in the aforementioned examples according to the invention were used. It was determined that the catalyst supports according to the invention are superior here, since the nitrogen doping has a dramatic, positive influence on the selectivities for the target products. In addition, decomposition of the products (e.g. in the case of Ru/C vs Ru/N—C) is significantly slowed/reduced by means of the nitrogen doping of the carbon support.

1-12. (canceled)

13. A method for chemically converting sugars or sugar alcohols into polyols/glycols, comprising:
converting sugars or sugar alcohols by hydrogenolysis in the presence of a catalyst comprising at least one metal and on a carbon support, and
using a nitrogen-doped carbon support as a catalyst support.

14. The method of claim 13, wherein, in a two-stage process, firstly a sugar is hydrogenated to give a sugar alcohol and thereafter the sugar alcohol is converted into polyols in a second step by means of hydrogenolysis.

15. The method of claim 13, including converting, by hydrogenation/hydrogenolysis, a C6 sugar or a C6 sugar alcohol or a C5 sugar or a C5 sugar alcohol into polyols/glycols.

16. The method of claim 13, wherein a nitrogen-doped activated carbon or nitrogen-doped carbon black is used as the catalyst support.

17. The method of claim 16, wherein a carbon support is used as catalyst, the surface of which has been doped with nitrogen atoms by reductive methods.

18. The method of claim 17, wherein said reductive methods uses ammonia and/or nitrogen and/or hydrogen.

19. The method of claim 13, wherein nitrogen-doped carbon nanotubes are used as the catalyst support.

20. The method of claim 19, wherein the carbon nanotubes are cylindrical carbon hollow bodies having a diameter of 0.4 to 100 nm which were additionally doped with nitrogen during the production thereof.

21. The method of claim 13, wherein the catalyst comprises one or more metals selected from the group comprising:
Ru, Pt, Ni, Os, Rh, Ir, Pd, and also Au, Ni, Cu, Fe and Co.

22. The method of claim 13, wherein a base is used as co-catalyst.

23. The method of claim 22, wherein the base is an alkali metal hydroxide or an alkaline earth metal hydroxide.

24. The method of claim 23, wherein the base is selected from the group comprising: (NaOH), KOH, LiOH, Mg(OH)₂, Ca(OH)₂, Sr(OH)₂ and Ba(OH)₂.

25. The method of claim 13, wherein the conversion is effected at a reaction temperature in the range from 20° C. to approximately 400° C.

26. The method of claim 13, wherein the conversion is effected at a reaction temperature in the range between 170° C. to approximately 200° C.

27. The method of claim 13, wherein the hydrogenolysis is effected at a hydrogen pressure in the range from approximately 1 bar to approximately 300 bar.

28. The method of claim 13, wherein the hydrogenolysis is effected at a hydrogen pressure in the range from 50 bar to approximately 80 bar.
